(19) United States
(12) Patent Application Publication
(10) Pub. No.: US 2009/0157008 A1
(43) Pub. Date: Jun. 18, 2009

(54) STERILE KIT CONTAINING SILICONE GEL FOR STEREOTAXY

(76) Inventor: Geraldo Sergio Farinazzo Vitral, Juiz de Fora (BR)

Correspondence Address: CANTOR COLBURN, LLP, 20 Church Street, 22nd Floor, Hartford, CT, 06103, US

(21) Appl. No.: 12/279,800

(22) PCT Filed: Dec. 7, 2006
(86) PCT No.: PCT/BR06/00266

§ 371 Date: Dec. 10, 2008

(30) Foreign Application Priority Data
Feb. 17, 2006 (BR) 8600225-2

(51) Int. Cl. A61M 5/31

(52) U.S. Cl. 604/187

(57) ABSTRACT

particularly a kit (K), which consists basically of a syringe (1), filled with silicone gel (2) (PMDS), which is supplied with or without a needle (3), and is appropriate for the stereotaxy procedure, showing an excellent radiological contrast and macroscopic locator in a histopathological test. The kit is completely sterile, without any type of negative or harmful interference in the nuclear medicine techniques, radiological localization of wounds and routine histological tests, and is packed in a pre-shaped recipient (4) or in a plastic blister pack (5). The kit may contain other components for use, such as gloves, cotton, gauze and similar (not demonstrated), everything made of disposable material.

This Utility Model Patent application relates to an original “SILICON-BASED DISPOSABLE STERILE KIT FOR STEREOTAXY”, particularly to a kit, which basically consists of two pieces, i.e. a syringe, which is pre-filled with silicone gel for radiological contrast, and is supplied with or without a needle, appropriate for the test to be performed; and is duly packed in a box or plastic film. The model in question points out because it uses polidimetilsyloxane, the peculiar characteristics of which are proved to be efficient in the elaboration of tests and/or procedures that need radiological contrast, such as for example the localization of impalpable or hidden wounds.

Stereotaxy is a radiological technique, which has been already widely used in the pre-surgical marking of impalpable neoplastic tissue wounds.

In the present STATE OF THE ART, as an infra-radiological control of confirmation of the area marked by the stereotaxy, iodined contrasts are used, which in spite of providing an adequate contrast on a radiological film, present some disadvantages resulting from their quick dispersion and/or absorption by human or animal tissues. Among these disadvantages we shall point out the following most common ones:
Image without exact delimitation, without sharpness (blurry);
Diffusion and absorption by the tissue within a short period of time that can make the procedure unfeasible;
Risk of allergic-anaphylactic reactions in iodine-sensitive patients;
Impossibility of identification of the marked area, in the macroscopic test of the surgical piece by the pathologist.

Having all these problems in mind, the inventor, after numberless researches, created the “SILICON-BASED DISPOSABLE STERILE KIT FOR STEREOTAXY”, which is a set consisting of a syringe that is pre-filled with silicone gel (polidimetilsyloxane—PDMS), and is aimed at replacing the iodine containing contrast. The kit preferentially contains a needle specific for this kind of procedure that has specific characteristics and does not present any problems of negative interference in the nuclear medicine techniques, radiological localization of impalpable wounds and routine histological tests. The kit in question can be also supplied without the aforementioned needle.

The new kit has the following main advantages, among many others:
Excellent radiological contrast;
Adequate and precise delimitation of the injection area, i.e. the area marked by the stereotaxy;
Reduction of occurrence of possible allergic-anaphylactic reaction in the patient;
Lack of local risk due to the complete excision of the marker;
Blocking of reflux of the radiological medicine after the injection, avoiding this way the radioactive contamination of the skin and the needle course in the tissue;
Adequate and exact macroscopic localization of the wound site by the pathologist that increases the diagnostic efficiency during the microscopic exam.

Dyes and other substances may be added to the silicone, either inert or not, and other supplements, such as gauze, gloves, cotton, antiseptic, anesthetic and similar materials can be also used.

In this way, a kit with exclusive characteristics is invented, and due to the use of silicone gel, which is completely disposable and duly packed, the product assures reliability, agility, accuracy, safety and low cost to the medical field professionals.

The kit will become more fully understood from the accompanying drawings, which are given by way of illustration only, and thus are not limitative of the present invention, and in which:

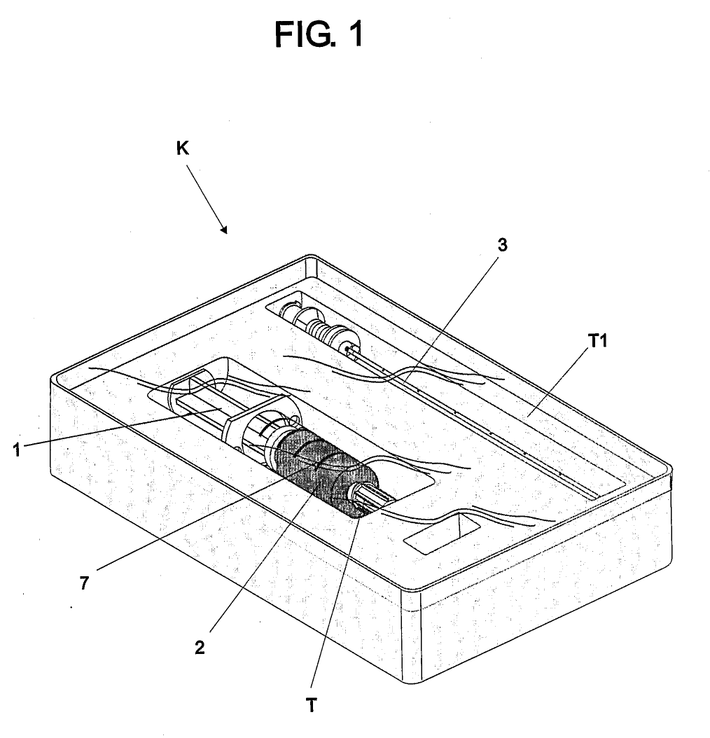

FIG. 1: Perspective view of the components of the kit, supplied together with a needle, packed in a pre-shaped package;

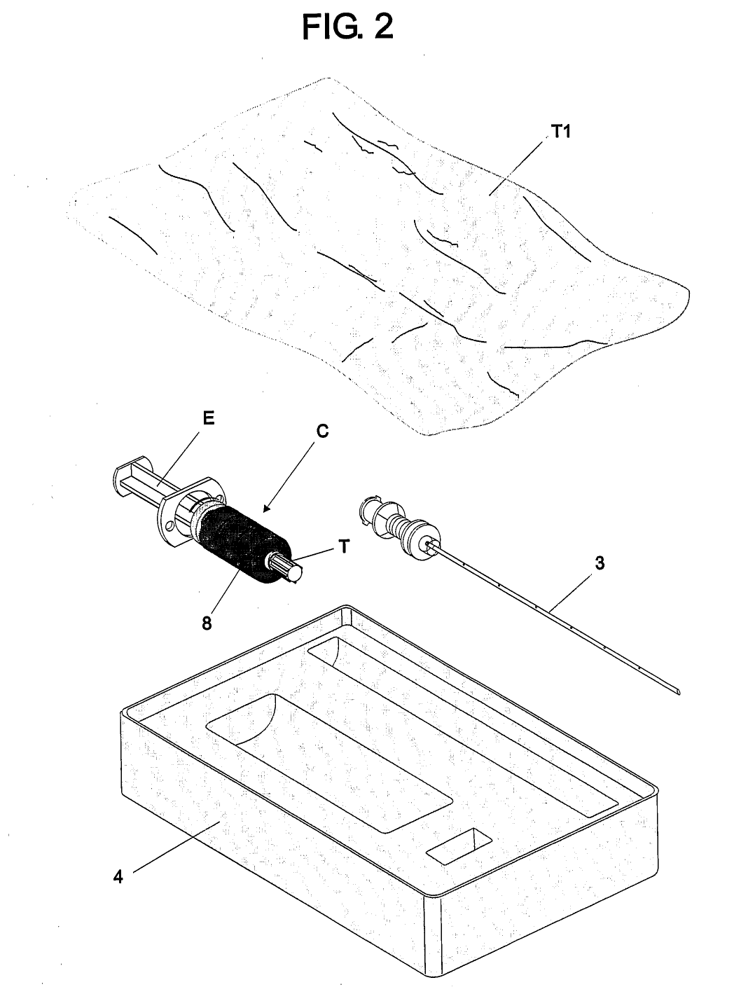

FIG. 2: Perspective exploded view of the kit, supplied together with a needle;

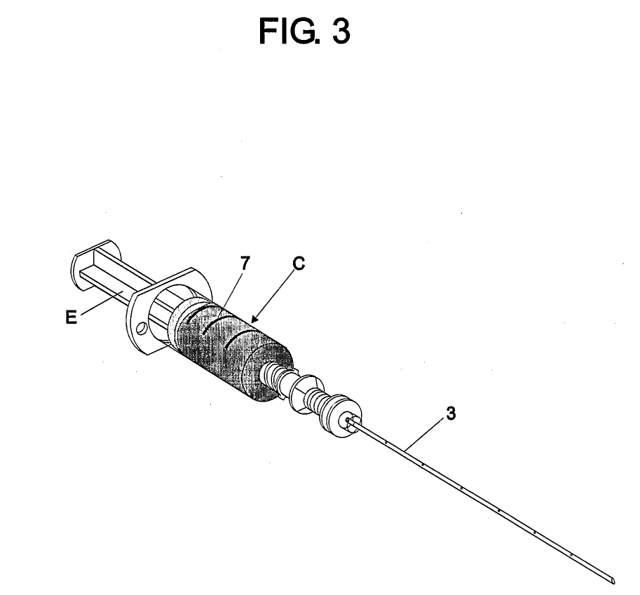

FIG. 3: Perspective view of the components of the kit, supplied together with an assembled needle;

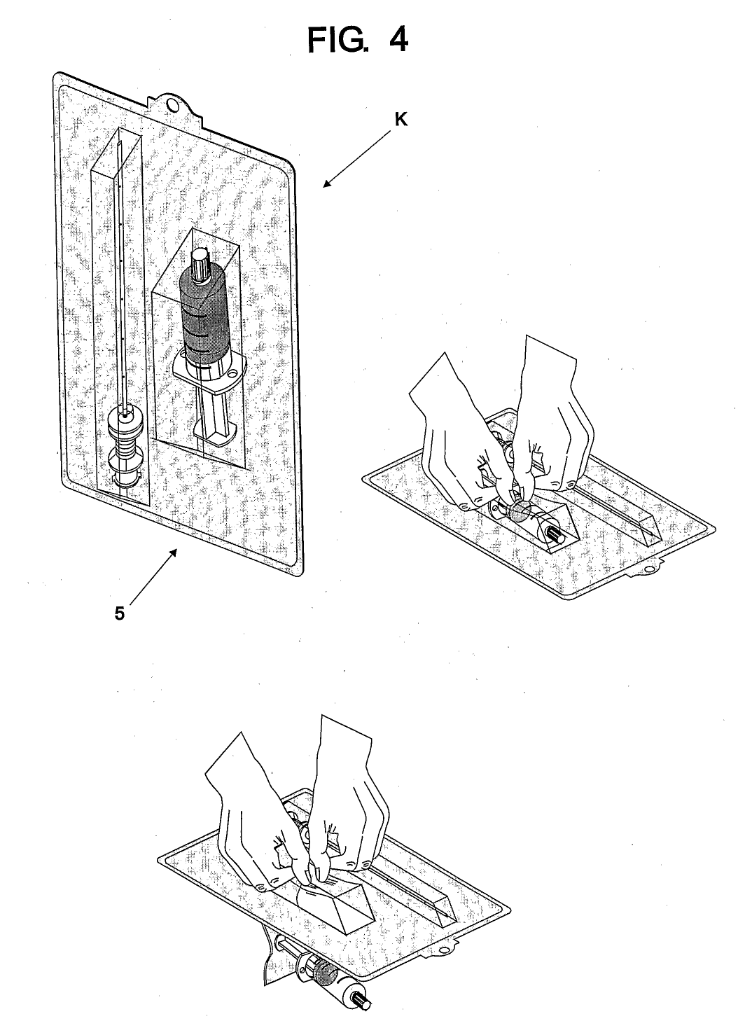

FIG. 4: Perspective view of the components of the kit, supplied together with a needle, packed in a plastic package;

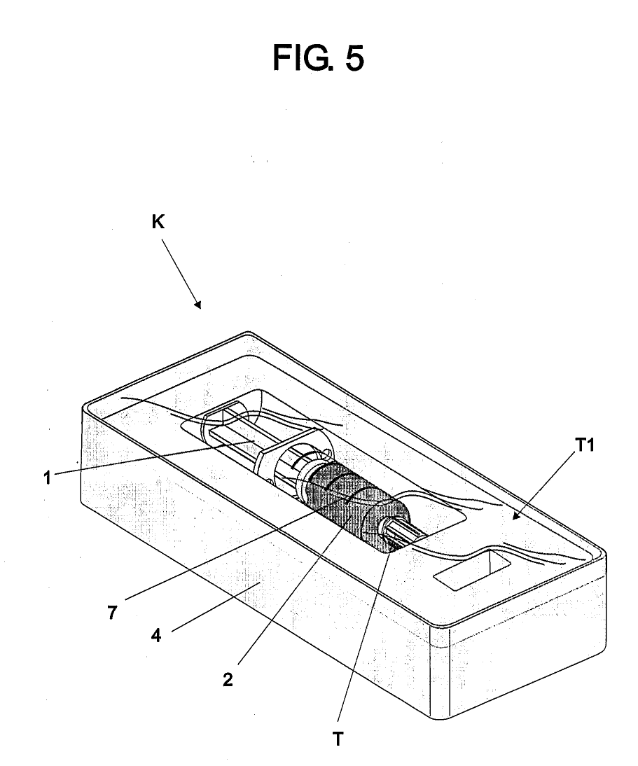

FIG. 5: Perspective view of the components of the kit, without needle, packed in a pre-shaped package;

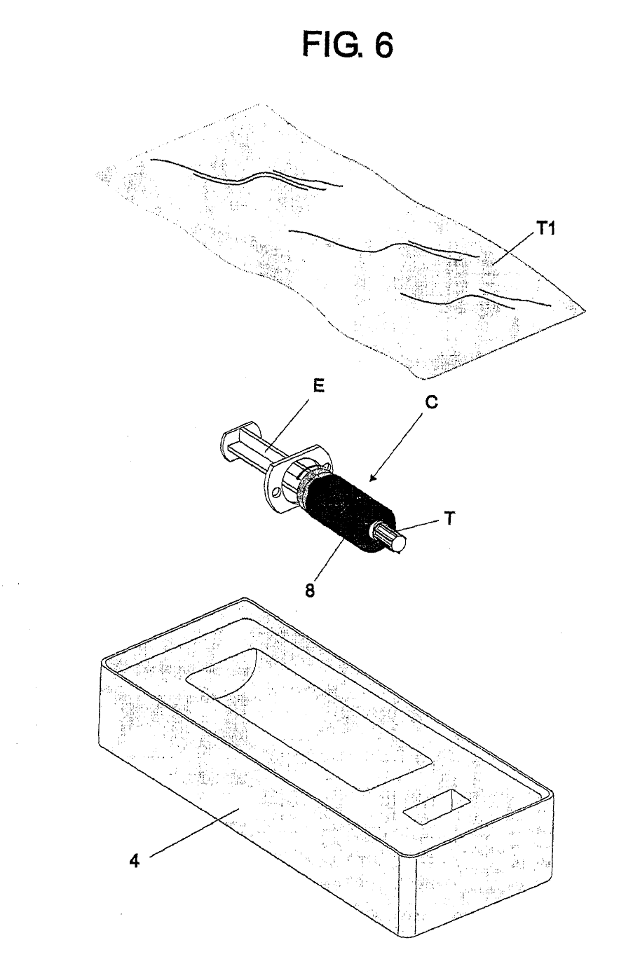

FIG. 6: Perspective exploded view of the kit without needle;

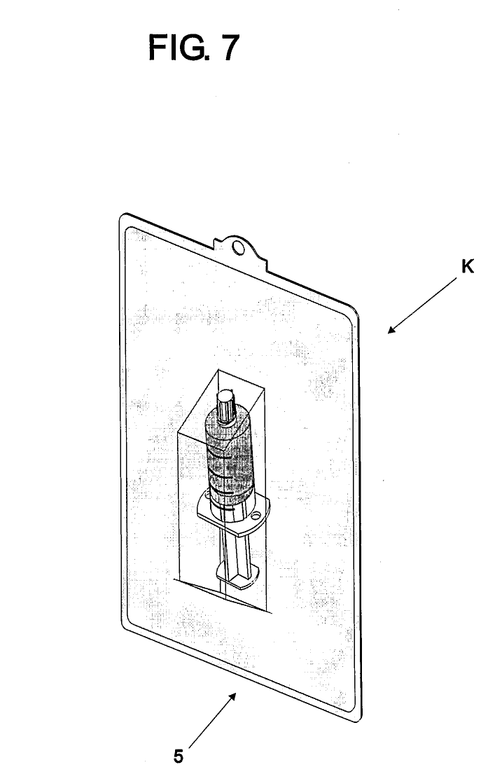

FIG. 7: Perspective view of the components of the kit without needle, packed in a plastic package.

The “SILICON-BASED DISPOSABLE STERILE KIT FOR STEREOTAXY”, object to this Utility Model Patent application, consists essentially of a kit (K), which basically contains a syringe (1), filled with silicone gel (2) (PDMS), supplied with or without a needle (3), appropriate for the procedure of stereotaxy that shows an excellent radiological contrast and macroscopic localization in a histopathological test, and is completely sterile, without any kind of negative or harmful interference in the nuclear medicine techniques, radiological localization of the wounds and histological routine tests, and is packed in a pre-shaped recipient (4) or in a plastic blister pack (5). The kit may contain other objects, such as gloves, cotton, gauze, and similar (not demonstrated), everything made of disposable material.

More preferentially, the kit (K) in question is composed of a syringe (1) of conventional operation, in which the piston (E) is displaced in the body (C) of the referred to syringe (1), which is duly marked (7) and supplied with an identification bar code (8). The fitting end of the needle (3), which is adequate for the procedure, is preferentially “luer-lock” type, or any other. The kit (K) points out because the referred to syringe (1) is pre-filled with silicone gel (2), more specifically, with polidimetilsyloxane. When the syringe (1) is not in use, i.e. remains in the package (5), it remains hermetically sealed due to the also conventional closure cap (T).

The kit (K) in question may be packed in a pre-shaped recipient (4) coated with a film (Ti), or in a plastic blister pack (5). Both packages are completely efficient, and can be accompanied by other components for use, such as cotton, gauze, gloves, antiseptic and anesthetic materials, etc. (not represented).

In a constructive variance, the kit (K) could also contain a syringe (1) without a needle, pre-filled with silicone gel (2), more specifically with polidimetilsyloxane, and when the syringe (1) is not in use, i.e. remains in the package (5), it remains hermetically sealed due to the also conventional closure cap (T).

The alternative kit (K) in question can be also packed in a pre-shaped recipient (4) coated with a film (Ti) or in a plastic blister pack (5). Both are completely efficient, and can be also supplied together other components for use, such as cotton, gauze, gloves, antiseptic and anesthetic materials, etc. (not represented).

If the same invention concept is maintained, dyes and other substances, either inert or not, may be added to the silicone gel (2).

1) “SILICON-BASED DISPOSABLE STERILE KIT FOR STEREOTAXY”, characterized by a kit (K) consisting of a syringe (1), the needle (3) fitting end of which is adequate to the procedure and is preferentially “luer-lock” type, or any other. The kit points out because the referred to syringe (1) is pre-filled with silicone gel (2), more specifically with polidimetilsyloxane, which when not in use, i.e. remains in the package (5), remains hermetically sealed due to the also conventional closure cap (8).

2) “SILICON-BASED DISPOSABLE STERILE KIT FOR STEREOTAXY”, in accordance with claim 1, is characterized by the package (4), which is pre-shaped (5), coated with a film (T1), or a plastic blister pack (5), and the referred to kit (K) can be also supplied with other components for use, such as cotton, gauze, gloves, antiseptic and anesthetic materials, etc. (not represented).

3) “SILICON-BASED DISPOSABLE STERILE KIT FOR STEREOTAXY”, in accordance with claim 1, is characterized by the kit (K), which can alternatively consist only of the syringe (1), which is pre-filled with the silicone gel (2), more specifically with polidimetilsyloxane. When the syringe (1) is not in use, i.e. remains in the package (5), it remains hermetically sealed due to the also conventional closure cap (T).

4) “SILICON-BASED DISPOSABLE STERILE KIT FOR STEREOTAXY”, in accordance with claim 3, is characterized by the alternative kit (K), is packed in a pre-shaped (4) recipient, coated with a film (T1), or in a plastic blister pack (5). Both are completely efficient, and can be supplied together with other components for use, such as cotton, gauze, gloves, antiseptic and anesthetic materials, etc. (not represented).

5) “SILICON-BASED DISPOSABLE STERILE KIT FOR STEREOTAXY”, in accordance with claim 1 and 3, is characterized by the fact that dyes or other substances, either inert or not, can be added to the silicone gel (2).
